(19) United States
(12) Patent Application Publication
(10) Pub. No.: US 2011/0195900 A1
(43) Pub. Date: Aug. 11, 2011

(54) PEPTIDIC PTH RECEPTOR AGONISTS

(75) Inventors: Claudio D. Schteingart, San Diego, CA (US); Javier Sueiras-Diaz, La Jolla, CA (US)

(73) Assignee: FERRING B.V., Hoofddorp (NL)

(21) Appl. No.: 13/059,622

(22) PCT Filed: Aug. 19, 2009
(86) PCT No.: PCT/US2009/054342

§ 371 Date: Feb. 17, 2011

Related U.S. Application Data
(60) Provisional application No. 61/090,062, filed on Aug. 19, 2008.

(51) Int. Cl. A61K 38/16; A61P 5/18; A61P 19/08; A61P 19/10; A61P 17/06; C07K 14/00

(52) U.S. Cl. 514/11.8; 514/16.7; 514/16.9; 514/18.6; 514/21.3; 530/324

(57) ABSTRACT

Novel peptidic pth receptor agonists and methods of using the same are provided.

### CROSS-REFERENCE TO RELATED APPLICATIONS

This application claims the benefit of U.S. provisional application 61/090,062 filed on Aug. 19, 2008, the entire disclosure of which is incorporated by reference in it entirety for all purposes.

The instant application contains a Sequence Listing which has been submitted in ASCII format via EFS-Web on Feb. 17, 2011 and is hereby incorporated by reference in its entirety. Said ASCII copy, created on Sep. 15, 2009, is named 15718_US_Sequence_Listing.txt and is 40 kb in size.

### FIELD OF THE INVENTION

The present invention relates to novel compounds, pharmaceutical compositions comprising these, use of the compounds for the manufacture of medicaments for inter alia treatment of diseases including osteoporosis, as well as to methods for treatment of said conditions, wherein said compounds are administered.

### BACKGROUND

PTH-1 receptor agonists, such as PTH (1-34), are known to be effective in the treatment of osteoporosis, as described by Neer, R. M., et al., in N. Engl. J. Med. 344 (2001) 1434-41. PTH-1 receptor agonists have also been shown to be effective in the acceleration of fracture healing, as suggested by Puzas, J. E., in J. Am. Acad. Orthop. Surg. 14 (2006) S145-51, and described by Manabe, T., in Bone 40 (2007) 1475-82, and the acceleration of cancellous bone healing, as described by Nozaka K., in Bone 42 (2008) 90-97. Such PTH-1 receptor agonists have also been shown to be effective in the treatment of hypoparathyroidism as described by Winer K. K., in J. Clin. Endocrinol. Metab. 10 (1998) 3480-6; the treatment of psoriasis, as described by Whitfield, J. F., in J. Cell. Biochem. 2 (2004) 251-6; and the stimulation of surgical implant fixation as described by Skripitz, R., in J. Orthop. Res. 6 (2005) 1266-70.

It has also been shown that once-daily injections of PTH (1-34), also known as intermittent injections, are superior to continuous infusion; see for example Neer, R. M. in N. Engl. J. Med. 344 (2001) 1434-41.

US 2006-0058230 A1 discloses improved PTH-1 receptor agonist analogues: bovine PTH (1-34) analogues that are primarily modified with one or more β- and β-homo-amino acids at the 16, 17, 18, 19 and 20 positions; and human PTH (1-34) analogues that are primarily modified with one or more natural amino acids between the 23 and 31 positions.

Schievano et al. in Biopolymers 70 (2003) 534-547 discloses PTH-1 receptor agonist analogues: human PTH (1-34) analogues modified with one or more β- and β-homo-amino acids at the 17, 18 and 19 positions together with Nle, Nal and Tyr at the 8, 23 and 34 positions.

Modification at the 16-20 positions with β- and β-homo-amino acids, such as the examples shown above, may provide improved selectivity; however, potency at the PTH-1 receptor may be reduced due to modification at these positions, see table 1 in Schievano et al.

### SUMMARY OF THE INVENTION

The present invention provides PTH-1 receptor agonist analogues that display improved selectivity for the PTH-1 receptor over the PTH-2 receptor whilst retaining a comparable or having improved potency when compared to PTH (1-34) potency (agonist activity) at the PTH-1 receptor.

According to the present invention in a first aspect there is provided a compound having the formula (I) (SEQ ID NO: 56):

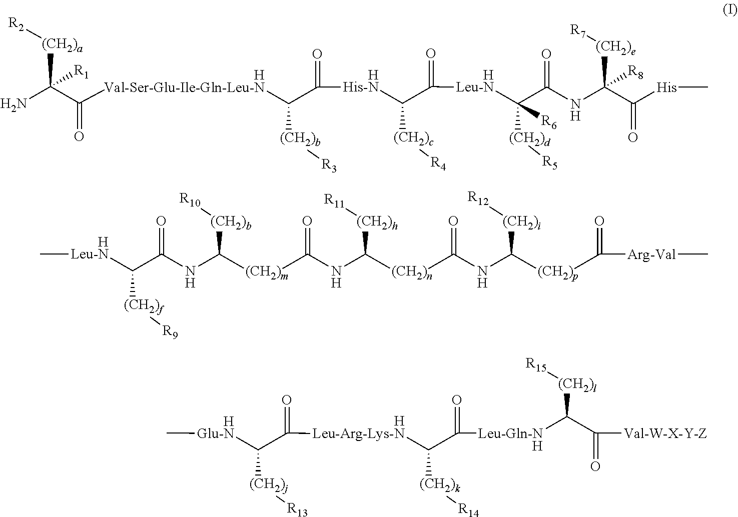

wherein:
R₁, R₆ and R₈ are independently H or alkyl;
R₂ is H or OH;
R₃ is H or O-alkyl;
R₄, R₅, R₇ and R₉ are independently selected from the group consisting of H, OH, NH₂, CO₂H and CONH₂;
R₁₀ is selected from the group consisting of H, OH, CONH₂, NHC(NH)NH₂ and aryl;
R₁₁ and R₁₂ are independently selected from the group consisting of H, alkyl, OH, NH₂, O-alkyl, CO₂H, CONH₂, aryl and NHC(NH)NH₂;
R₁₃ is optionally substituted 1-naphthyl or 2-naphthyl;
R₁₄ is NH₂ or alkyl;

R₁₅ is selected from the group consisting of OH, CO₂H, heteroaryl and NHC(NH)NH₂;
a, c, d and j are independently 1 or 2;
b is 2 or 3;
e and k are independently 1 or 4;
f is selected from the group consisting of 1, 2 and 4;
g is selected from the group consisting of 0, 1, 2, and 3, with the proviso that when R₁₀ is other than H, g is not 0;
l is selected from the group consisting of 1, 2 and 3;
h and i are independently selected from the group consisting of 0, 1, 2, 3 and 4, with the proviso that when R¹¹ is other than H, h is not 0 and when R¹² is other than H, i is not 0;
m is 0 or 1;
n and p are independently selected from the group consisting of 0, 1 and 2;
W, X and Y are independently present or absent and if present are independently selected from the group consisting of histidine, asparagine, glutamine, glutamic acid, phenylalanine and lysine;
Z is NR₁₆R₁₇ or OH, wherein R₁₆ and R₁₇ are each independently selected from the group consisting of hydrogen, alkyl, heteroalkyl, cycloalkyl, aryl, heteroaryl and aralkyl and wherein R¹⁶ and R¹⁷ are optionally joined to form a 4- to 7-membered ring which may be a heterocycle or a heteroaryl;
alkyl is an optionally substituted C₁₋₆ straight chain alkyl or optionally substituted C₃₋₈ branched chain alkyl;
aryl is an optionally substituted mono- or bi-cyclic aromatic carbocyclic ring system;
aralkyl is an alkyl group which has as a substituent an aryl group; and
heteroaryl is an optionally substituted aromatic heterocyclic five- or six-membered ring system;
   and pharmaceutically acceptable salts thereof.

In an alternate embodiment Z is NH₂ or OH in formula I.

In another embodiment, R₁₄ may be CH(CH₃)₂ and k is 1 in formula I.

In another embodiment, R₁ is CH₃, R₂ is H and a is 1 in formula I.

In another embodiment R₁₆ is H in formula I.

In another embodiment R₁₀ is H or phenyl in formula I.

In another embodiment R₁₁ is selected from the group consisting of H, CH(CH₃)₂, CH(CH₃)CH₂CH₃, OH, OCH₃, CONH₂, phenyl and NHC(NH)NH₂ in formula I.

In another embodiment R₁₂ is selected from H, CH(CH₃)₂, CH(CH₃)CH₂CH₃, CH(OH)CH₃, OH, NH₂, CO₂H, CONH₂, phenyl, 4-hydroxyphenyl and NHC(NH)NH₂ in formula I.

In another embodiment j is 1 in formula I.

In another embodiment one or more of m, n and p is 1 in formula I.

In another embodiment, the compound comprises 31 or 34 amino acids.

In another embodiment, the compound of the invention is selected from the group consisting of:

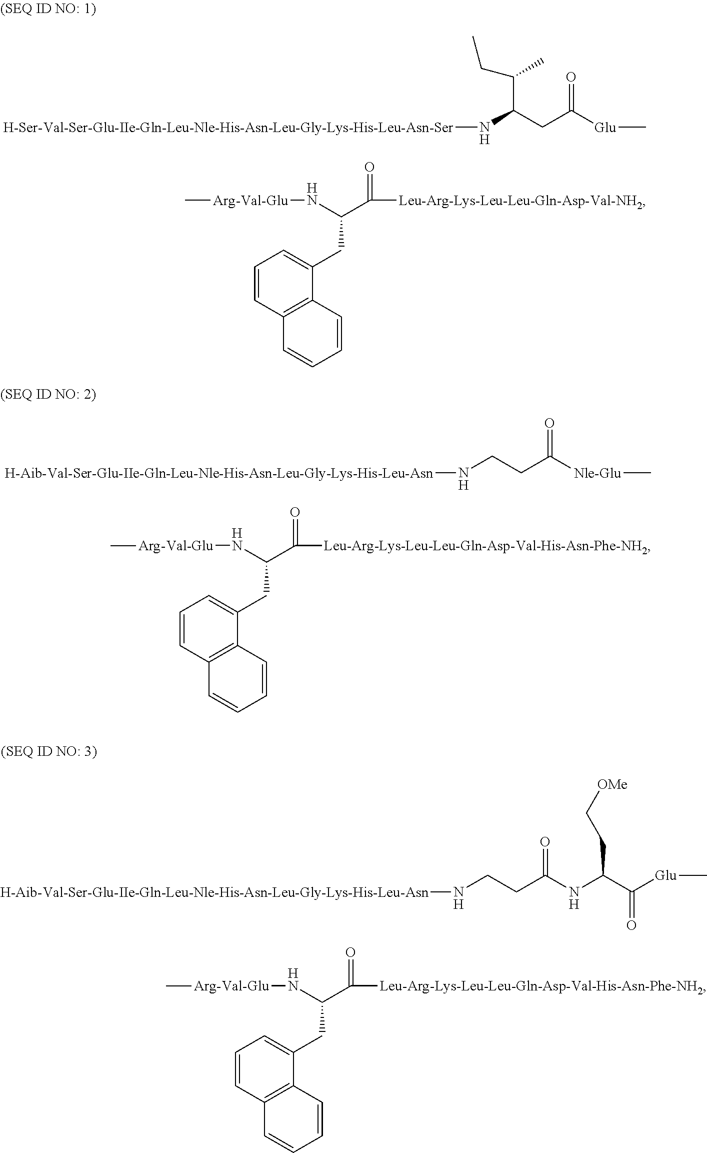

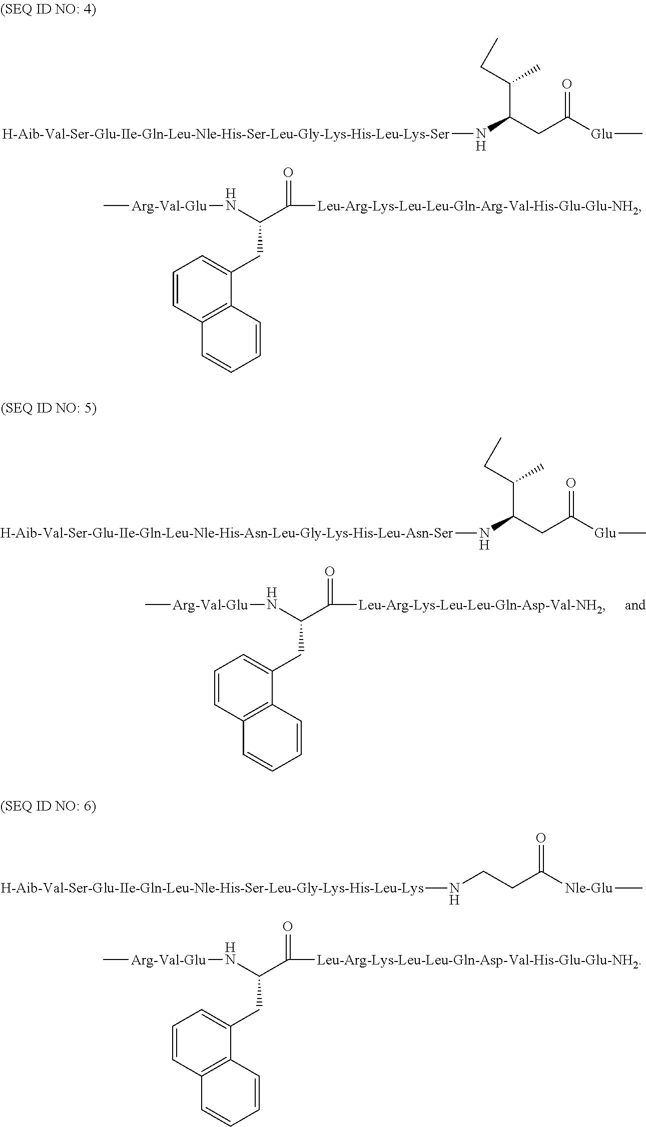

Also provided are compounds for use as a pharmaceutical.

Also provided are pharmaceutical compositions comprising compounds of the invention as active ingredient in association with a pharmaceutically acceptable adjuvant, diluent or carrier.

Also provided are uses of compounds of the invention the manufacture of a medicament for one of the group consisting of: treatment of one of the group consisting of psoriasis, hypoparathyroidism, and a disease of the bone; enhancement of bone growth; acceleration of bone growth; treatment of a bone fracture; and stimulating fixation of a surgical implant to a bone.

In one embodiment, the medicament is for the treatment of a disease of the bone selected from the group consisting of osteoporosis, rickets, osteomalacia and Paget's disease.

In one embodiment, the medicament is for the treatment of a cancellous bone fracture.

Also provided are methods of treatment of one of the group consisting of psoriasis, hypoparathyroidism, and a disease of the bone; enhancement of bone growth; acceleration of bone growth; treatment of a bone fracture; and stimulating fixation of a surgical implant to a bone, the method comprising administering to an animal, including human, patient of a therapeutically effective amount of a compound of the invention.

In one embodiment the method treats a disease of the bone selected from the group consisting of osteoporosis, rickets, osteomalacia and Paget's disease.

In one embodiment the method treats a cancellous bone fracture.

### DETAILED DESCRIPTION

For the purposes of the present invention, the following terminology is used.

C₁₋₆ straight chain alkyl denotes all alkyl chain groups having from one to six carbon atoms, including any number therebetween.

C₃₋₈ branched chain alkyl denotes all branched alkyl groups containing three to eight carbon atoms, including iso-, sec-, and tent-configurations.

Heteroaryl is selected from aromatic heterocyclic five- or six-membered ring systems.

A five-membered heteroaromatic ring system is a monocyclic aromatic ring system having five ring atoms, wherein 1, 2, 3 or 4 ring atoms are independently selected from N, O and S. Exemplary ring systems include imidazolyl, thiazolyl, furyl, pyrazolyl, triazolyl and optionally substituted imidazolyl, thiazolyl, furyl, pyrazolyl and triazolyl.

A six-membered heteroaromatic ring system is a monocyclic aromatic ring system having six ring atoms, wherein 1, 2, 3 or 4 ring atoms are independently selected from N, O and S. Exemplary ring systems include pyridyl, pyrimidyl, pyrazinyl and optionally substituted pyridyl, pyrimidyl and pyrazinyl.

Exemplary mono- and bi-cyclic aromatic carbocyclic ring systems include phenyl, naphthyl and optionally substituted phenyl and optionally substituted naphthyl.

Optionally substituted means that there may be one or more substituent group present on a specified moiety. The substituent moieties may be for example fluorine (F), chlorine (Cl) and bromine (Br) atoms and alkyl, hydroxy (—OH), alkoxy (—O-alkyl), alkylthio (—S-alkyl), cyano (CN), amino (NH₂), amido (CONH₂), carboxyl (CO₂H) and C₁₋₆ alkyl ester (CO₂-alkyl).

Examples of pharmaceutically acceptable salts include acid addition salts, e.g. a salt formed by reaction with hydrohalogen acids such as hydrochloric acid and mineral acids, such as sulphuric acid, phosphoric acid and nitric acid, as well as aliphatic, alicyclic, aromatic or heterocyclic sulphonic or carboxylic acids such as formic acid, acetic acid, propionic acid, succinic acid, glycolic acid, lactic acid, malic acid, tartaric acid, citric acid, benzoic acid, ascorbic acid, maleic acid, hydroxymaleic acid, pyruvic acid, p-hydroxybenzoic acid, embonic acid, methanesulphonic acid, ethanesulphonic acid, hydroxyethanesulphonic acid, halobenzenesulphonic acid, trifluoroacetic acid, trifluoromethanesulphonic acid, toluenesulphonic acid and naphthalenesulphonic acid.

The abbreviations used are:

| Abbreviation | Conventional Name |
|---|---|
| Bu | butyl - alkyl residues may be further denoted as n |
| | (normal, i.e. unbranched), i (iso), s (sec) and |
| | t (tertiary) |
| CH₃CN | acetonitrile |
| DCM | dichloromethane |
| DIPEA | N,N-diisopropylethylamine |
| DMF | N,N-dimethylformamide |
| Fmoc | 9-fluorenylmethoxycarbonyl |
| h | hour(s) |
| HATU | N-[(dimethylamino)-1H-1,2,3-triazolo[4,5-b]pyridine- |
| | 1-ylmethylene]-N-methylmethanaminium |
| | hexafluorophosphate N-oxide |
| HPLC | high performance liquid chromatography |
| hPTH | human parathyroid hormone |
| hPTH-1 receptor | human parathyroid hormone receptor 1 |
| hPTH-2 receptor | human parathyroid hormone receptor 2 |
| hPTHR-1 | human parathyroid hormone receptor 1 |
| hPTHR-2 | human parathyroid hormone receptor 2 |
| μg | microgram |
| Pal | peptide amide linker [5-(4-Fmoc-aminomethyl-3,5- |
| | dimethoxy-phenoxy)valeric acid] |
| PEG | polyethylene glycol |
| PS | polystyrene |
| tBu | tert-butyl |
| tBuOH | tert-butylalcohol |
| TFA | trifluoroacetic acid |
| TIPS | triisopropylsilane |
| TG | tentagel |
| Trt | trityl [triphenylmethyl, (C₆H₅)₃C—] |

Unless otherwise specified L-amino acids were used and conventional amino acid terminology is adhered to.

Some examples of conventional amino acid terminology include:

| Abbreviation | Conventional Name |
|---|---|
| Aib | 2-aminoisobutyric acid |
| Nle | Norleucine |
| Ala | Alanine |
| Ser | Serine |
| Glu | Glutamine |
| Lys | Lysine |
| β-hAla | β-homo-Alanine |
| β-hPhe | β-homo-Phenylalanine |
| β-Ala | β-Alanine |
| β-hIle | β-homo-Isoleucine |
| h-Ser(OMe) | homo-Serine methyl ether |
| β-hArg | β-homo-Arginine |
| β-hGlu | β-homo-Glutamic acid |
| 1-Nal | 1-Naphthylalanine |
| 2-Nal | 2-Naphthylalanine |
| Leu | Leucine |
| His | Histidine |
| Arg | Arginine |

According to the present invention in a first aspect there is provided a compound having the formula (I) (SEQ ID NO: 57):

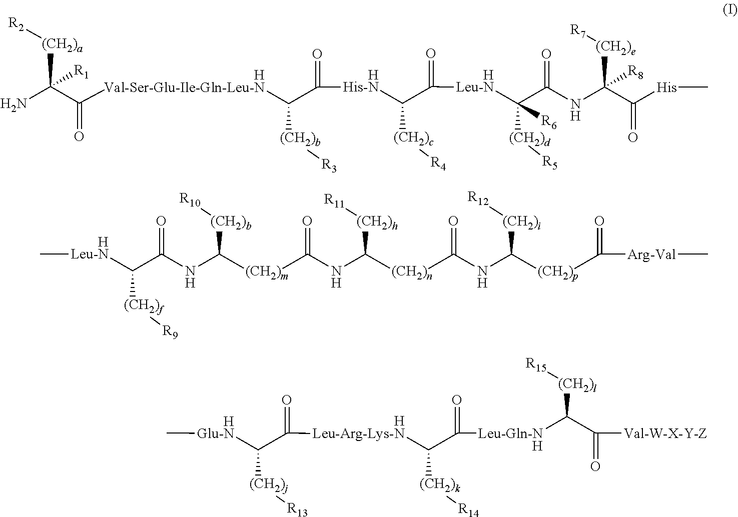

wherein:
R₁, R₆ and R₈ are independently H or alkyl;
R₂ is H or OH;
R₃ is H or O-alkyl;
R₄, R₅, R₇ and R₉ are independently selected from the group consisting of H, OH, NH₂, CO₂H and CONH₂;
R₁₀ is selected from the group consisting of H, OH, CONH₂, NHC(NH)NH₂ and aryl;
R₁₁ and R₁₂ are independently selected from the group consisting of H, alkyl, OH, NH₂, O-alkyl, CO₂H, CONH₂, aryl and NHC(NH)NH₂;
R₁₃ is optionally substituted 1-naphthyl or 2-naphthyl;
R₁₄ is NH₂ or alkyl;
R₁₅ is selected from the group consisting of OH, CO₂H, heteroaryl and NHC(NH)NH₂;
a, c, d and j are independently 1 or 2;
b is 2 or 3;
e and k are independently 1 or 4;
f is selected from the group consisting of 1, 2 and 4;
g is selected from the group consisting of 0, 1, 2, and 3, with the proviso that when R₁₀ is other than H, g is not 0;
l is selected from the group consisting of 1, 2 and 3;
h and i are independently selected from the group consisting of 0, 1, 2, 3 and 4, with the proviso that when R¹¹ is other than H, h is not 0 and when R¹² is other than H, i is not 0;
m is 0 or 1;
n and p are independently selected from the group consisting of 0, 1 and 2;
W, X and Y are independently present or absent and if present are independently selected from the group consisting of histidine, asparagine, glutamine, glutamic acid, phenylalanine and lysine;
Z is NR₁₆R₁₇ or OH, wherein R₁₆ and R₁₇ are each independently selected from the group consisting of hydrogen, alkyl, heteroalkyl, cycloalkyl, aryl, heteroaryl and aralkyl and wherein R¹⁶ and R¹⁷ are optionally joined to form a 4- to 7-membered ring which may be a heterocycle or a heteroaryl;
alkyl is an optionally substituted C₁₋₆ straight chain alkyl or optionally substituted C₃₋₈ branched chain alkyl;
aryl is an optionally substituted mono- or bi-cyclic aromatic carbocyclic ring system;
aralkyl is an alkyl group which has as a substituent an aryl group; and heteroaryl is an optionally substituted aromatic heterocyclic five- or six-membered ring system;
   and pharmaceutically acceptable salts thereof.

In an alternate embodiment Z is NH₂ or OH.

In yet another embodiment, R₁₄ is CH(CH₃)₂ and k is 1. This results in Leu at position 27.

In yet another embodiment, R₁ is CH₃, R₂ is H and a is 1. This results in Aib at position 1.

In yet another embodiment, R₁₃ is 1-naphthyl and j is 1. This results in 1-Nal in position 23.

In another embodiment, one or more of m, n and p is 1. This results in a β- or β-homo-amino acid at the 17 position if m=1, at the 18 position if n=1 and at the 19 position if p=1.

The applicant has found that, when 1-Nal is in the 23 position, and/or Leu is in the 27 position; optionally Aib is in the 1 position; and/or one or more of m, n and p is 1, compounds of the present invention show improved PTH-1 receptor selectivity over the PTH-2 receptor. Additionally, compounds of the invention show improved potency when compared to the potency of PTH (1-34).

R₁₀ may be H when m is 1 and g is 0 or 1.

R₁₀ may be phenyl when m is 1 and g is 1.

R₁₁ may be H when n is 1 and h is 0 or 1.

R₁₁ may be H when n is 0 and h is 4.

R₁₁ may be CH(CH₃)CH₂CH₃ when n is 1 and h is 0.

R₁₁ may be OCH₃ when n is 0 and h is 2.

R₁₁ may be phenyl when n is 1 and h is 1.

R₁₂ may be NH₂ when p is 1 and i is 4.

R₁₂ may be H when p is 1 and i is 0.

R₁₂ may be CO₂H when p is 1 and i is 2.

In a further example, the compound may be one of the following:

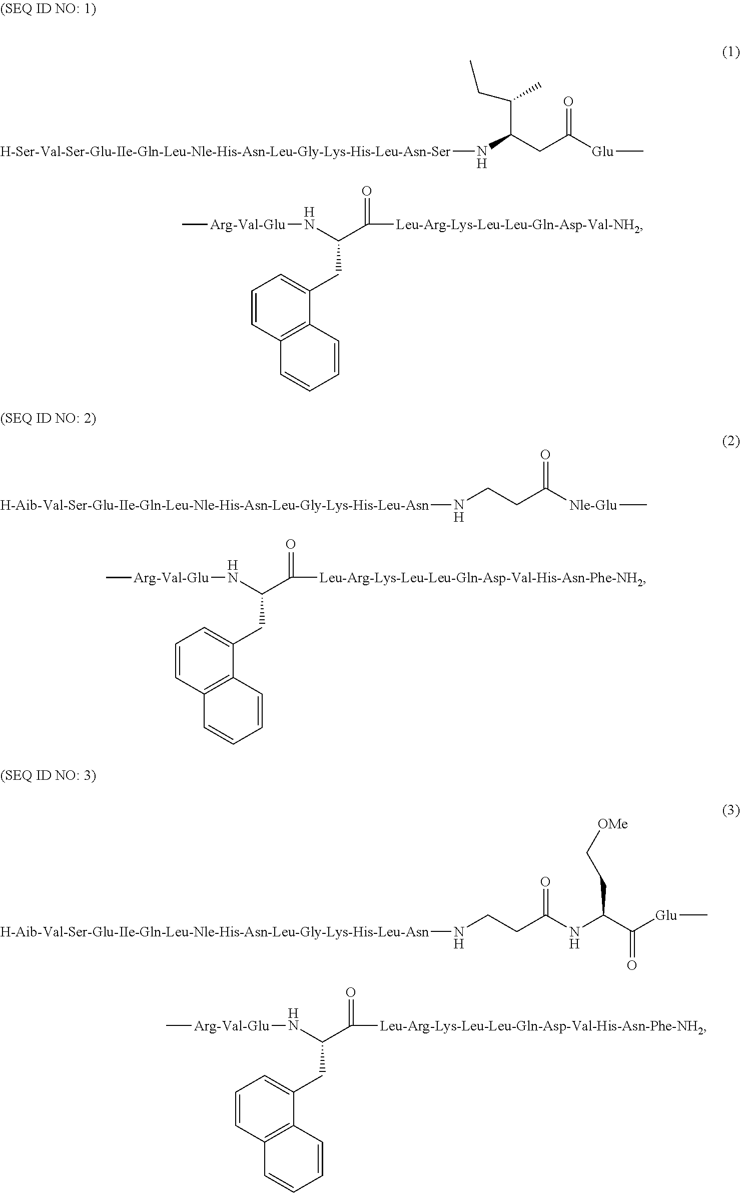

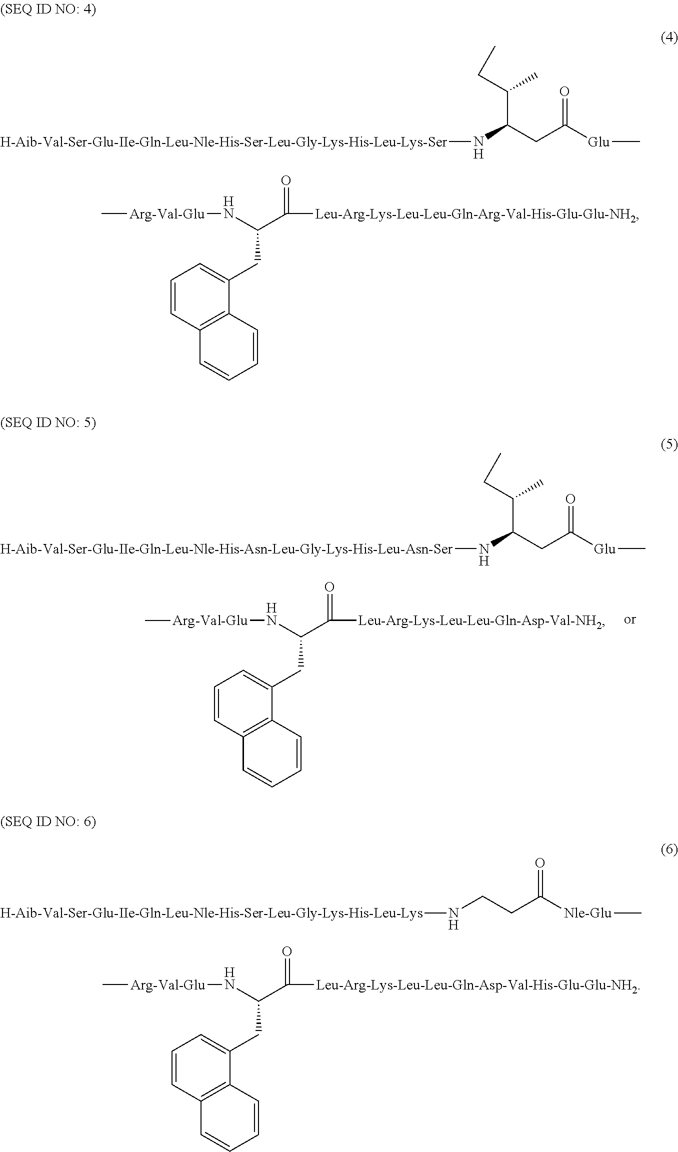

### Pharmaceutical Compositions

According to the present invention in a further aspect there is provided the use of a compound of formula (I), as defined herein, as a pharmaceutical.

According to the present invention in a further aspect there is provided a pharmaceutical composition comprising a compound of formula (I), as defined herein, as active ingredient in association with a pharmaceutically acceptable adjuvant, diluent or carrier.

The pharmaceutical composition may be adapted for oral, intravenous, topical, interperitoneal, nasal, buccal, intraocular, intra-aural, sublingual, intramuscular and subcutaneous administration and for administration via the respiratory tract e.g. in the form of an aerosol or an air-suspended fine powder. The composition may thus for instance be in the form of tablets, capsules, powders, microparticles, granules, syrups, suspensions, solutions, transdermal patches or suppositories.

It should be noted that the pharmaceutical composition according to the present invention may include two or more of the above defined compounds.

The pharmaceutical composition may optionally comprise e.g. at least one further additive selected from a disintegrating agent, binder, lubricant, flavouring agent, preservative, colourant and any mixture thereof. Examples of such and other additives are found in ‘Handbook of Pharmaceutical Excipients’; Ed. A. H. Kibbe, 3^{rd} Ed., American Pharmaceutical Association, USA and Pharmaceutical Press UK, 2000.

The pharmaceutical composition may be adapted for parenteral administration, e.g. by injection. Preferred liquid carriers, for injectable solutions, include by way of example and without limitation water, saline, aqueous dextrose and glycols. The injectable aqueous formulation FORTEO® (teriparatide) is an example of a suitable pharmaceutical formulation applicable also for the inventive compounds disclosed herein.

In a further aspect the present invention provides the use of a compound as outlined above for the manufacture of a medicament for treatment of diseases including psoriasis, hypoparathyroidism and diseases of the bone including, but not limited to, osteoporosis, rickets, osteomalacia and Paget's disease, treatment for enhancing and/or accelerating bone growth, for example treatment of bone fracture including cancellous bone fracture, and treatment of patients with surgical implant in order to stimulate fixation to the bone.

In another aspect the invention provides methods for treatment of diseases including psoriasis, hypoparathyroidism, diseases of the bone including, but not limited to, osteoporosis, rickets, osteomalacia and Paget's disease, treatment for enhancing and/or accelerating bone growth, for example treatment of bone fracture including cancellous bone fracture, treatment of patients with surgical implant in order to stimulate fixation to the bone, wherein said method comprises administering to an animal, including human, patient a therapeutically effective amount of a compound as outlined above.

As used herein ‘treatment’ means the alleviation of symptoms, postponement of the onset of the disease and/or the cure of the disease when a compound of the invention is administered in a suitable dose. As used herein ‘treatment of diseases of the bone’ means that, upon administration of a compound of the invention, the patient's bone density increases, remains the same, or does not decrease as rapidly as it would without the administration of a compound of the invention.

The typical dosage of the compounds according to the present invention varies within a wide range and will depend on various factors such as the individual needs of each patient and the route of administration. The dosage may be administered once daily or more frequently than once daily, e.g. intermittently. The dosage administered, for example, is generally within the range of 0.01-1000 μg, e.g. from 1 to 200 μg per day, e.g. by subcutaneous injection. A physician of ordinary skill in the art will be able to optimise the dosage to the situation at hand.

### EXAMPLES

α-Amino acid derivatives were purchased from commercial providers (Bachem, Novabiochem and Peptides International) and β- and β-homo-amino acid derivatives were purchased from commercial providers (PepTech Corporation). Resins were purchased from commercial providers (Applied Biosystems and Novabiochem). All additional reagents, chemicals and solvents were purchased from Sigma-Aldrich, Fluka and Acros Organics.

### Synthesis

The compounds herein were synthesised by standard methods in solid phase peptide chemistry utilising Fmoc methodology and an Applied Biosystems Pioneer ‘Peptide Synthesis System’ continuous-flow automated peptide synthesizer with cycle protocols specified by Applied Biosystems.

Preparative HPLC was performed on a Waters Delta Prep LC 4000 using a PrepPack cartridge Delta-Pack C18, 300 Å, 15 μm, 47×300 mm at a flow rate of 100 ml/min. Analytical reverse phase HPLC was performed on a Waters 600 liquid chromatograph using a Vydac column 218TP54, C18 (5 μm, 4.6×250 mm) at a flow rate of 2 ml/min. Final compound analysis was performed on a reverse phase HPLC on a Vydac C18 column (5 μm, 2.1×250 mm at a flow rate of 0.3 ml/min.

Unless otherwise provided, all reactions were performed at room temperature. The following standard reference literature provides further guidance on general experimental set up, as well as on the availability of required starting material and reagents:
Kates, S. A., Albericio, F., Eds., Solid Phase Synthesis: A Practical Guide, Marcel Dekker, New York, Basel, 2000;
Greene, T. W., Wuts, P. G. M., Protective Groups in Organic Synthesis, John Wiley Sons Inc., 2^{nd} Edition, 1991;
Stewart, J. M., Young, J. D., Solid Phase Synthesis, Pierce Chemical Company, 1984;
Bisello, et al., J. Biol. Chem. 1998, 273, 22498-22505; and
Merrifield, J. Am. Chem. Soc. 1963, 85, 2149-2154.
Chang and White P. D., ‘Fmoc Solid Phase Peptide Synthesis: a Practical Approach’, Oxford University Press, Oxford, 2000.

Purity of the synthesised peptide may be determined by analytical reverse phase HPLC. Structural integrity of the peptides may be confirmed using amino acid analysis and electrospray mass spectrometry.

N-α-Fmoc-Pal-PEG-PS resin (Applied Biosystems, Foster City, Calif.) was used as starting material for C-terminal carboxylamide peptides. N-α-Fmoc protected amino acid attached to NovaSyn® TGA resin (Novabiochem, San Diego, Calif.) was used as starting material for peptides with a free carboxyl C-terminus.

The following protecting groups were utilized to protect the given amino acid side chain functional groups: 2,2,5,7,8-pentamethylchroman-6-sulfonyl (Arg); t-butyl group (Glu, Asp, Ser, Thr and Tyr); trityl group (H is, Gln and Asn); t-butoxycarbonyl group (Lys).

All coupling of Fmoc-protected amino acids were mediated with HATU/DIPEA in DMF. Single couplings of 35 minutes with a 4-fold excess of activated Fmoc-protected amino acids were used during the synthesis. Each coupling reaction was followed by treatment with a solution of 5% acetic anhydride and 6% 2,4-lutidine in DMF. Removal of the Fmoc protecting group was performed by a six minute flow-wash of the peptide resin with 30% piperidine in DMF.

Upon completion of the peptide synthesis and deprotection of the terminal Fmoc protecting group, the peptides were washed with DCM and dried in vacuo. The peptides were treated with TFA/H₂O/TIPS (44:5:0.75) (25 ml) for 90 mins in order to remove the side-chain protecting groups with concomitant cleavage of the peptide from the resin. The peptide was filtered, precipitated with cool t-butylmethylether, centrifuged (the solid was retained), washed with t-butylmethylether, re-dissolved in acetonitrile-H₂O (70:30), lyophylised and purified by preparative HPLC.

Purification of the peptides was performed with preparative HPLC. Each crude peptide was purified with either buffer system P or buffer system C, or P followed by C. The fractions with a purity exceeding 93%, determined by reverse-phase analytical HPLC, were reloaded onto the column and eluted with buffer T to provide trifluoroacetate salts. The fractions were pooled and lyophilized.

TABLE 1
Buffer compositions

| Buffer | Component A | Component B |
|---|---|---|
| P | 0.25M Triethylammonium | 80% acetonitrile in 20% A |
| | Phosphate (TEAP) | |
| | (pH 2.3 or pH 5.4) | |
| C | 0.1M Triethylammonium | 80% acetonitrile in 20% A |
| | Perchlorate (TEAClO₄) (pH 2.3) | |
| T | 0.1% Trifluoroacetic acid (TFA) | 80% acetonitrile in 20% A |

Table 2 describes the amino acids in specific positions in terms of the nomenclature utilised above.

### Additional Syntheses

Alternatively, peptide amides can be prepared by assembling the peptide on an appropriate resin by solid phase methods, cleaving the fully protected peptide containing a free C-terminal acid from the resin, coupling it to the desired amine in solution, followed by general deprotection and purification. Methods are well known in the art, for example as described in M. Goodman, A. Felix, L. Moroder, C. Toniolo, Editors; Synthesis of Peptides and Peptidomimetics, Houben-Weyl, Vols E22a to E22e, Georg Thime Verlag, 2004.

In another embodiment, secondary amides, such as compounds of the present invention wherein R₁₇ is hydrogen and R₁₆ is not hydrogen, may be made on solid phase as described in “Backbone Amide Linker (BAL) Strategy for Solid-Phase Synthesis of C-Terminal-Modified and Cyclic Peptides,” Knud J. Jensen, Jordi Alsina, Michael F. Songster, Josef Vagner, Fernando Albericio, and George Barany, Journal of the American Chemical Society 1998 120 (22), 5441-5452 and “An Alkanesulfonamide “Safety-Catch” Linker for Solid-Phase Synthesis,” Bradley J. Backes and Jonathan A. Ellman, The Journal of Organic Chemistry 1999 64 (7), 2322-2330.

In yet another embodiment, tertiary or cyclic amides, such as compounds of the present invention where in neither R₁₆ nor R₁₇ are hydrogen and R₁₆ and R₁₇ are optionally joined to form a ring, may be made on solid phase as described in “Structure-Based Design and Synthesis of High Affinity Tripeptide Ligands of the Grb2-SH2 Domain,” Pascal Furet, Brigitte Gay, Giorgio Caravatti, Carlos García-Echeverría, Joseph Rahuel, Joseph Schoepfer, and Heinz Fretz, Journal of Medicinal Chemistry 1998 41 (18), 3442-3449.

TABLE 2
Key to amino acid nomenclature

| | | Amino Acid | | |
|---|---|---|---|---|
| Amino Acid | Position | as it appears in table 3 | Claim Nomenclature | |
| Aib | 1 | Aib¹ | R₁ = CH₃, R₂ = H | a = 1 |
| Ala | 1 | Ala¹ | R₁ = H, R₂ = H | a = 1 |
| Nle | 8 | Nle⁸ | R₃ = H | b = 4 |
| Ser | 10 | Ser¹⁰ | R₄ = OH | c = 1 |
| Glu | 10 | Glu¹⁰ | R₄ = CO₂H | c = 2 |
| Aib | 12 | Aib¹² | R₅ = H, R₆ = CH₃ | d = 1 |
| Aib | 13 | Aib¹³ | R₇ = H, R₈ = CH₃ | e = 1 |
| Glu | 16 | Glu¹⁶ | R₉ = CO₂H | f = 2 |
| Ser | 16 | Ser¹⁶ | R₉ = OH | f = 1 |
| Lys | 16 | Lys¹⁶ | R₉ = NH₂ | f = 4 |
| β-hAla | 17 | β-hAla¹⁷ | R₁₀ = H | g = 1, m = 1 |
| β-hPhe | 17 | β-hPhe¹⁷ | R₁₀ = phenyl | g = 1, m = 1 |
| β-Ala | 17 | β-Ala¹⁷ | R₁₀ = H | g = 0, m = 1 |
| β-Ala | 18 | β-Ala¹⁸ | R₁₁ = H | h = 0, n = 1 |
| β-hIle | 18 | β-hIle¹⁸ | R₁₁ = CH(CH₃)CH₂CH₃ | h = 0, n = 1 |
| Nle | 18 | Nle¹⁸ | R₁₁ = H | h = 4, n = 0 |
| h-Ser(OMe) | 18 | h-Ser(OMe)¹⁸ | R₁₁ = OCH₃ | h = 2, n = 0 |
| β-hAla | 18 | β-hAla¹⁸ | R₁₁ = H | h = 1, n = 1 |
| β-hPhe | 18 | β-hPhe¹⁸ | R₁₁ = phenyl | h = 1, n = 1 |
| β-hArg | 19 | β-hArg¹⁹ | R₁₂ = NHC(NH)NH₂ | i = 3, p = 1 |
| β-hAla | 19 | β-hAla¹⁹ | R₁₂ = CH₃ | i = 1, p = 1 |
| β-hGlu | 19 | β-hGlu¹⁹ | R₁₂ = CO₂H | i = 2, p = 1 |
| 1-Nal | 23 | 1-Nal²³ | R₁₃ = 1-naphthyl | j = 1 |
| 2-Nal | 23 | 2-Nal²³ | R₁₃ = 2-naphthyl | j = 1 |
| Leu | 27 | Leu²⁷ | R₁₄ = CH(CH₃)₂ | k = 1 |
| His | 30 | His³⁰ | R₁₅ = imidazol-2-yl | l = 1 |
| Ser | 30 | Ser³⁰ | R₁₅ = OH | l = 1 |
| Arg | 30 | Arg³⁰ | R₁₅ = NHC(NH)NH₂ | l = 3 |
| Glu | 30 | Glu³⁰ | R₁₅ = CO₂H | l = 2 |

### Biological Assays

### In Vitro Reporter Gene Assays:

Agonist activity of compounds on the human PTH-1 receptor was determined in a reporter gene assay by transiently transfecting HEK-293 cells with plasmids encoding human PTH-1 receptor under control of the SV40 promoter and a luciferase reporter gene under control of cAMP responsive elements (CRE). See Himmler et al, J. Recept. Res. 13 (1993) 79-94, for further guidance on this assay. Transfected cells were exposed to serial dilutions of compounds for 4-5 hours followed by lysis of cells, determination of luciferase activity, and determination of compound efficacies and EC₅₀ values through non-linear regression.

PTH (1-34) was used as an internal control in each experiment and compounds were tested in at least three independent experiments. To determine selectivity, compounds were tested in luciferase-based transcriptional reporter gene assays expressing the human PTH-2 receptor. The results of these experiments are shown in tables 3 and 4.

Table 3 comprises compounds of the formula (I) prepared by the example Synthesis procedure and biological data.

TABLE 3

| SEQ | | hPTHR-1 | hPTHR-2 | hPTHR-2/ |
|---|---|---|---|---|
| ID | Compound Synthesised and Tested as described above | EC₅₀ (nM) | EC₅₀ (nM) | hPTHR-1 |
| 1 | [Nle⁸, β-hIle¹⁸, 1-Nal²³, Leu²⁷] hPTH-(1-31)-NH₂ | 0.09 | 32 | 339 |
| 2 | [Aib¹, Nle^{8,18}, β-Ala¹⁷, 1-Nal²³, Leu²⁷] hPTH (1-34)-NH₂ | 0.06 | 31 | 566 |
| 3 | [Aib¹, Nle⁸, β-Ala¹⁷, hSer(OMe)¹⁸, 1-Nal²³, Leu²⁷] hPTH (1- | 0.06 | 49 | 766 |
| | 34)-NH₂ | | | |
| 4 | [Aib¹, Nle⁸, Ser¹⁰, Lys¹⁶, β-hIle¹⁸, 1-Nal²³, Leu²⁷, Arg³⁰, | 0.04 | 4 | 110 |
| | Glu³³, Glu³⁴] hPTH (1-34)-NH₂ | | | |
| 5 | [Aib¹, Nle⁸, β-hIle¹⁸, 1-Nal²³, Leu²⁷] hPTH (1-31)-NH₂ | 0.05 | 18 | 387 |
| 6 | [Aib¹, Nle^{8,18}, Ser¹⁰, Lys¹⁶, β-Ala¹⁷, 1-Nal²³, Leu²⁷, Arg³⁰, | 0.03 | 7 | 267 |
| | Glu³³, Glu³⁴] hPTH (1-34)-NH₂ | | | |
| 7 | [Nle^{8,18}, β-Ala¹⁹, 1-Nal²³] hPTH (1-34)-NH₂ | 0.21 | 11 | 52 |
| 8 | [Nle⁸, β-hIle¹⁸, 1-Nal²³] hPTH (1-34)-OH | 0.21 | 26 | 124 |
| 9 | [Nle⁸, β-hIle¹⁸, 1-Nal²³, Leu²⁷] hPTH (1-34)-OH | 0.14 | 23 | 158 |
| 10 | [Nle⁸, β-hIle¹⁸, 1-Nal²³, Leu²⁷] hPTH (1-31)-OH | 0.09 | 76 | 811 |
| 11 | [Nle^{8,18}, β-Ala¹⁹, 1-Nal²³, Leu²⁷] hPTH-(1-34)-NH₂ | 0.26 | 18 | 69 |
| 12 | [Ala¹, Nle^{8,18}, β-Ala¹⁷, 2-Nal²³] hPTH (1-34)-NH₂ | 0.22 | 95 | 430 |
| 13 | [Aib¹, Nle^{8,18}, β-Ala¹⁷, 2-Nal²³] hPTH (1-34)-NH₂ | 0.22 | 46 | 213 |
| 14 | [Ala¹, Nle^{8,18}, β-Ala¹⁷, 1-Nal²³, Leu²⁷] hPTH (1-34)-NH₂ | 0.20 | 89 | 449 |
| 15 | [Nle^{8,18}, β-Ala¹⁷, 1-Nal²³, Leu²⁷] hPTH (1-34)-NH₂ | 0.12 | 47 | 389 |
| 16 | [Nle⁸, β-Ala¹⁷, hSer(OMe)¹⁸, 1-Nal²³, Leu²⁷] hPTH (1-34)- | 0.13 | 93 | 719 |
| | NH₂ | | | |
| 17 | [hSer(OMe)^{8,18}, β-Ala¹⁷, 1-Nal²³, Leu²⁷] hPTH(1-34)-NH₂ | 0.88 | >270 | >300 |
| 18 | [Nle⁸, β-hAla¹⁸, 1-Nal²³, Leu²⁷] hPTH (1-34)-NH₂ | 0.22 | 61 | 281 |
| 19 | [Nle⁸, β-hIle¹⁸, 1-Nal²³, Leu²⁷] hPTH (1-34)-NH₂ | 0.14 | 32 | 224 |
| 20 | [Nle^{8,18}, β-hGlu¹⁹, 1-Nal²³, Leu²⁷] hPTH (1-34)-NH₂ | 0.32 | 29 | 91 |
| 21 | [Nle⁸, Glu¹⁶, β-hIle¹⁸, 1-Nal²³, Leu²⁷] hPTH (1-34)-NH₂ | 0.16 | 37 | 234 |
| 22 | [Nle⁸, Ser¹⁶, β-hIle¹⁸, 1-Nal²³, Leu²⁷] hPTH (1-34)-NH₂ | 0.19 | 20 | 101 |
| 23 | [Nle⁸, Ser¹⁰, β-hIle¹⁸, 1-Nal²³, Leu²⁷] hPTH (1-34)-NH₂ | 0.18 | 17 | 95 |
| 24 | [Nle⁸, Glu¹⁰, β-hIle¹⁸, 1-Nal²³, Leu²⁷] hPTH (1-34)-NH₂ | 0.13 | 3 | 24 |
| 25 | [Nle⁸, β-hIle¹⁸, 1-Nal²³, Leu²⁷, His³⁰] hPTH (1-34)-NH₂ | 0.18 | 13 | 72 |
| 26 | [Nle⁸, β-hIle¹⁸, 1-Nal²³, Leu²⁷, Ser³⁰] hPTH (1-34)-NH₂ | 0.19 | 19 | 98 |
| 27 | [Nle⁸, β-hIle¹⁸, 1-Nal²³, Leu²⁷, Glu³³] hPTH (1-34)-NH₂ | 0.12 | 41 | 344 |
| 28 | [Nle⁸, β-hIle¹⁸, 1-Nal²³, Leu²⁷, Lys³³] hPTH (1-34)-NH₂ | 0.15 | 32 | 209 |
| 29 | [Nle⁸, β-hIle¹⁸, 1-Nal²³, Leu²⁷, Glu³⁴] hPTH (1-34)-NH₂ | 0.12 | 57 | 474 |
| 30 | [Nle⁸, β-hIle¹⁸, 1-Nal²³, Leu²⁷, Lys³⁴] hPTH (1-34)-NH₂ | 0.10 | 17 | 173 |
| 31 | [Nle⁸, Aib^{12,13}, β-hIle¹⁸, 1-Nal²³, Leu²⁷] hPTH (1-34)-NH₂ | 0.25 | 34 | 138 |
| 32 | [Nle⁸, Aib¹², β-hIle¹⁸, 1-Nal²³, Leu²⁷] hPTH (1-34)-NH₂ | 0.23 | 13 | 55 |
| 33 | [Nle⁸, Aib¹³, β-hIle¹⁸, 1-Nal²³, Leu²⁷] hPTH (1-34)-NH₂ | 0.14 | 68 | 481 |
| 34 | [Nle⁸, Lys¹⁶, β-hIle¹⁸, 1-Nal²³, Leu²⁷] hPTH (1-34)-NH₂ | 0.08 | 30 | 373 |
| 35 | [Nle⁸, β-hIle¹⁸, 1-Nal²³, Leu²⁷, Arg³⁰] hPTH (1-34)-NH₂ | 0.18 | 7 | 40 |
| 36 | [Aib¹, Nle⁸, β-hIle¹⁸, 1-Nal²³, Leu²⁷] hPTH (1-34)-NH₂ | 0.04 | 16 | 372 |
| 37 | [Nle⁸, Lys¹⁶, β-hIle¹⁸, 1-Nal²³, Leu²⁷, Arg³⁰] hPTH (1-34)- | 0.14 | 14 | 97 |
| | NH₂ | | | |
| 38 | [Aib¹, Nle⁸, Ser¹⁰, Lys¹⁶, β-hIle¹⁸, 1-Nal²³, Leu²⁷, Arg³⁰, | 0.09 | 6 | 61 |
| | Glu³³] hPTH (1-34)-NH₂ | | | |
| 39 | [Aib¹, Nle⁸, Lys¹⁶, β-hIle¹⁸, 1-Nal²³, Leu²⁷, Arg³⁰, Glu³³] | 0.09 | 6 | 61 |
| | hPTH (1-34)-NH₂ | | | |
| 40 | [Nle⁸, Ser¹⁰, Lys¹⁶, β-hIle¹⁸, 1-Nal²³, Leu²⁷, Arg³⁰, Glu³³] | 0.12 | 8 | 69 |
| | hPTH(1-34)-NH₂ | | | |
| 41 | [Nle⁸, Lys¹⁶, β-hIle¹⁸, 1-Nal²³, Leu²⁷, Arg³⁰, Glu³³] hPTH (1- | 0.09 | 14 | 157 |
| | 34)-NH₂ | | | |
| 42 | [Aib¹, Nle⁸, β-hPhe¹⁸, 1-Nal²³, Leu²⁷ hPTH (1-34)-NH₂ | 0.06 | 5 | 88 |
| 43 | [Aib¹, Nle⁸, β-hArg¹⁸, 1-Nal²³, Leu²⁷] hPTH (1-34)-NH₂ | 0.05 | 16 | 312 |
| 44 | [Nle⁸, Ser¹⁰, Lys¹⁶, β-hIle¹⁸, 1-Nal²³, Leu²⁷, Arg³⁰, Glu³³, | 0.04 | 6 | 154 |
| | Glu³⁴] hPTH (1-34)-NH₂ | | | |
| 45 | [Aib¹, Nle⁸, Ser¹⁰, Lys¹⁶, β-hIle¹⁸, 1-Nal²³, Leu²⁷, Arg³⁰] | 0.09 | 6 | 72 |
| | hPTH (1-31)-NH₂ | | | |
| 46 | [Nle⁸, β-hPhe¹⁸, 1-Nal²³, Leu²⁷] hPTH (1-34)-NH₂ | 0.11 | 10 | 94 |
| 47 | [Aib¹, Nle⁸, hSer(OMe)¹⁸, β-hLys¹⁹, 1-Nal²³, Leu²⁷] hPTH (1- | 0.05 | 3 | 51 |
| | $$4)-NH₂ | | | |
| 48 | [Aib¹, Nle⁸, β-hPhe¹⁷, hSer(OMe)¹⁸, 1-Nal²³, Leu²⁷] hPTH-(1- | 0.10 | 117 | 1152 |
| | 34)-NH₂ | | | |
| 49 | [Nle⁸, Ser¹⁰, Lys¹⁶, β-hIle¹⁸, 1-Nal²³, Leu²⁷, Arg³⁰] hPTH (1- | 0.08 | 7 | 87 |
| | 31)-NH₂ | | | |
| 50 | [Aib¹, Nle⁸, Glu¹⁰, Lys¹⁶, β-hIle¹⁸, 1-Nal²³, Leu²⁷, Glu³⁰, | 0.04 | 1 | 22 |
| | Glu³³, Lys³⁴] hPTH(1-34)-NH₂ | | | |
| 51 | hPTH (1-34) | 0.11 | 4.31 | 39 |

hPTH (1-34) and hPTH (1-31) have the following amino acid sequences (where the C-terminus is CONH₂):

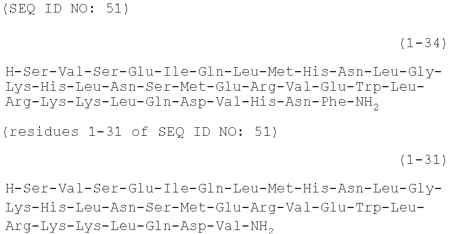

It is understood that SEQ ID NO: 51 is hPTH(1-34).

Conventional amino acid terminology well known in the art is used herein. A superscript following the three letter abbreviation of an amino acid's name denotes the position of the amino acid in the peptide sequence. For example ‘Nle⁸’ means that there is an Nle (norleucine) in the 8 position of the peptide sequence. All peptide sequences are written with the convention that the N-terminal amino acid is on the left and the C-terminal amino acid is on the right. Numbering starts at the N-terminus and proceeds towards the C-terminus. For example, SEQ ID NO: 2 [Aib¹, Nle^{8,18}, β-Ala¹⁷, 1-Nal²³, Leu²⁷] hPTH-(1-34)-NH₂ means a peptide having a sequence of hPTH in which specific amino acid residues in the wild type sequence have been substituted with alternative amino acids: the Ser residue in the 1 position has been replaced with Aib; the Met residues in the 8 and 18 positions have been replaced with Nle; the Ser residue in the 17 position has been replaced with β-Ala; the Trp residue in the 23 position has been replaced with 1-Nal; and the Lys residue in the 27 position has been replaced with Leu. As used herein, ‘hPTH(1-34)’ refers to a shortened human sequence of PTH (hPTH) having amino acids 1 to 34, as set forth in SEQ ID NO: 51.

The hPTH-(1-34)-NH₂ section of SEQ ID NO: 2 indicates that the C-terminus is an amide, i.e. CONH₂. Alternatively, the C-terminus may be a carboxylic acid, i.e. CO₂H; this is indicated by an OH at the right of the sequence, as shown by SEQ ID NO: 8.

Table 3 shows the potency of compounds disclosed herein at the hPTH-1 receptor. Improved potency when compared to PTH (1-34) can be seen, for example by SEQ ID NO: 1; SEQ ID NO: 6 has an EC₅₀ value of 0.03 nM at the hPTH-1 receptor compared to an EC₅₀ value of 0.11 nM for PTH (1-34).

Table 3 shows an improved selectivity at the hPTH-1 receptor over the hPTH-2 receptor by the compounds disclosed herein, when compared to PTH (1-34). Improved selectivity can be seen, for example by SEQ ID NO: 3. SEQ ID NO: 3 has a 766-fold selectivity for the hPTH-1 receptor over the hPTH-2 receptor. In comparison, PTH (1-34) shows a 38-fold selectivity for the hPTH-1 receptor over the hPTH-2 receptor.

TABLE 4
Comparison Data

| | | | | | Selectivity |
|---|---|---|---|---|---|
| | SEQ | | hPTHR-1 | hPTHR-2 | hPTHR-2/ |
| Group | ID | Compound Synthesised and Tested as described above | EC₅₀ (nM) | EC₅₀ (nM) | hPTHR-1 |
| A | 52 | [Nle⁸, β-hAla¹⁸, 2-Nal²³] hPTH (1-34)-NH₂ | 0.34 | 73 | 215 |
| | 18 | [Nle⁸, β-hAla¹⁸, 1-Nal²³, Leu²⁷] hPTH (1-34)-NH₂ | 0.22 | 61 | 281 |
| B | 53 | [Nle⁸, β-hIle¹⁸, 2-Nal²³] hPTH (1-34)-NH₂ | 0.25 | 42 | 170 |
| | 19 | [Nle⁸, β-hIle¹⁸, 1-Nal²³, Leu²⁷] hPTH (1-34)-NH₂ | 0.14 | 32 | 224 |
| C | 54 | [Nle⁸, β-hArg¹⁸, 2-Nal²³] hPTH (1-34)-NH₂ | 0.24 | 36 | 147 |
| | 43 | [Aib¹, Nle⁸, β-hArg¹⁸, 1-Nal²³, Leu²⁷] hPTH (1-34)-NH₂ | 0.05 | 16 | 312 |
| D | 55 | [Aib¹, Nle^{8,18}, β-Ala¹⁷, 1-Nal²³] hPTH (1-34)-NH₂ | 0.22 | 46 | 213 |
| | 2 | [Aib¹, Nle^{8,18}, β-Ala¹⁷, 1-Nal²³, Leu²⁷] hPTH (1-34)-NH₂ | 0.06 | 31 | 566 |
| E | 19 | [Nle⁸, β-hIle¹⁸, 1-Nal²³, Leu²⁷] hPTH (1-34)-NH₂ | 0.14 | 32 | 224 |
| | 36 | [Aib¹, Nle⁸, β-hIle¹⁸, 1-Nal²³, Leu²⁷] hPTH (1-34)-NH₂ | 0.04 | 16 | 372 |
| | 5 | [Aib¹, Nle⁸, β-hIle¹⁸, 1-Nal²³, Leu²⁷] hPTH (1-31)-NH₂ | 0.05 | 18 | 387 |

The following discussion of activity refers to the compounds in Table 4. Compounds listed in Table 4 are all compounds according to the invention.

Groups A and B show that the combination of 1-Nal²³ and Leu²⁷ improves both the potency of a compound at hPTH-1 receptor and selectivity of a compound for PTH-1 receptor over PTH-2 receptor, in comparison to the corresponding compounds with 2-Nal²³ and Lys²⁷.

Group C shows that the combination of Aib¹, 1-Nal²³ and Leu²⁷ improves both the potency of a compound at hPTH-1 receptor and selectivity of a compound for the PTH-1 receptor over the PTH-2 receptor, when compared to a corresponding analogue comprising Ser¹, 2-Nal²³ and Lys²⁷.

Group D shows that Leu²⁷ improves both the potency of a compound at the hPTH-1 receptor and selectivity of a compound for the PTH-1 receptor over the PTH-2 receptor, when compared to a corresponding analogue comprising Lys²⁷.

Group E shows that Aib¹ improves both the potency of a compound at the hPTH-1 receptor and selectivity of a compound for the PTH-1 receptor over the PTH-2 receptor, when compared to a corresponding analogue comprising Ser¹.

Group E also shows that comparable potency and selectivity may be retained for peptides with a 31 amino acid sequence.

The improved potency at the PTH-1 receptor of the compounds having formula (I) and described herein, and improved selectivity at the PTH-1 receptor over the PTH-1 receptor, show that they would for instance be useful in the safe and efficacious treatment of human beings with diseases such as psoriasis, hypoparathyroidism and diseases of the bone including, but not limited to, osteoporosis, rickets, osteomalacia and Paget's disease, treatment for enhancing and/or accelerating bone growth, for example treatment of bone fracture including cancellous bone fracture, and treatment of patients with surgical implant in order to stimulate fixation to the bone.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1. A compound having the formula (I) (SEQ ID NO: 56): 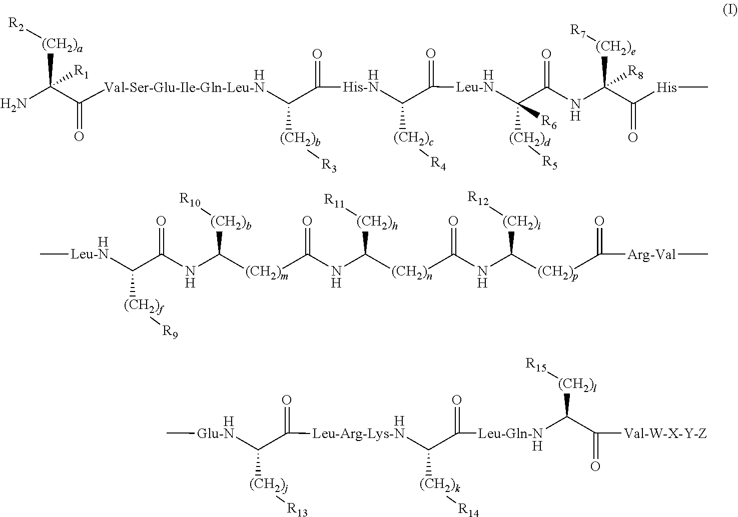
wherein:
R₁, R₆ and R₈ are independently H or alkyl;
R₂ is H or OH;
R₃ is H;
R₄, R₅, R₇ and R₉ are independently selected from the group consisting of H, OH, NH₂, CO₂H and CONH₂;
R₁₀ is selected from the group consisting of H, OH, CONH₂, NHC(NH)NH₂ and aryl;
R₁₁ and R₁₂ are independently selected from the group consisting of H, alkyl, OH, NH₂, CO₂H, CONH₂, aryl and NHC(NH)NH₂;
R₁₃ is an optionally substituted 1-naphthyl or an optionally substituted 2-naphthyl;
R₁₄ is NH₂ or alkyl;
R₁₅ is selected from the group consisting of OH, CO₂H, heteroaryl and NHC(NH)NH₂;
a, c, d and j are independently 1 or 2;
b is 2 or 3;
e and k are independently 1 or 4;
f is selected from the group consisting of 1, 2 and 4;
g is selected from the group consisting of 0, 1, 2, and 3, with the proviso that when R₁₀ is other than H, g is not 0;
l is selected from the group consisting of 1, 2 and 3;
h and i are independently selected from the group consisting of 0, 1, 2, 3 and 4, with the proviso that when R¹¹ is other than H, h is not 0 and when R¹² is other than H, i is not 0;
m is 0 or 1;
n and p are independently selected from the group consisting of 0, 1 and 2;
wherein one or more of m, n, and p is 1;
W, X and Y are independently present or absent and if present are independently selected from the group consisting of histidine, asparagine, glutamine, glutamic acid, phenylalanine and lysine;
Z is NR₁₆R₁₇ or OH, wherein R₁₆ and R₁₇ are each independently selected from the group consisting of hydrogen, alkyl, heteroalkyl, cycloalkyl, aryl, heteroaryl and aralkyl and wherein R¹⁶ and R¹⁷ are optionally joined to form a 4- to 7-membered ring which may be a heterocycle or a heteroaryl;
alkyl is an optionally substituted C₁₋₆ straight chain alkyl or optionally substituted C₃₋₈ branched chain alkyl;
aryl is an optionally substituted mono- or bi-cyclic aromatic carbocyclic ring system;
aralkyl is an alkyl group which has as a substituent an aryl group; and
heteroaryl is an optionally substituted aromatic heterocyclic five- or six-membered ring system;
and pharmaceutically acceptable salts thereof.

2. A compound according to claim 1, wherein Z is NH₂ or OH.

3. A compound according to claim 1, wherein k is 1 and R₁₄ is CH(CH₃)₂.

4. A compound according to claim 1, wherein R₁ is CH₃, R₂ is H and a is 1.

5. A compound according to claim 1 wherein R₁₆ is hydrogen.

6. A compound according to claim 1, wherein R₁₀ is selected from the group consisting of H and phenyl.

7. A compound according to claim 1, wherein R₁₁ is selected from H, CH(CH₃)₂, CH(CH₃)CH₂CH₃, OH, CONH₂, phenyl and NHC(NH)NH₂.

8. A compound according to claim 1, wherein R₁₂ is selected from H, CH(CH₃)₂, CH(CH₃)CH₂CH₃, CH(OH)CH₃, OH, NH₂, CO₂H, CONH₂, phenyl, 4-hydroxyphenyl and NHC(NH)NH₂.

9. A compound according to claim 1, wherein j is 1.

10. A compound according to claim 1, wherein the sequence comprises 31 or 34 amino acids.

11. A compound according to claim 1, wherein the compound is selected from the group consisting of:

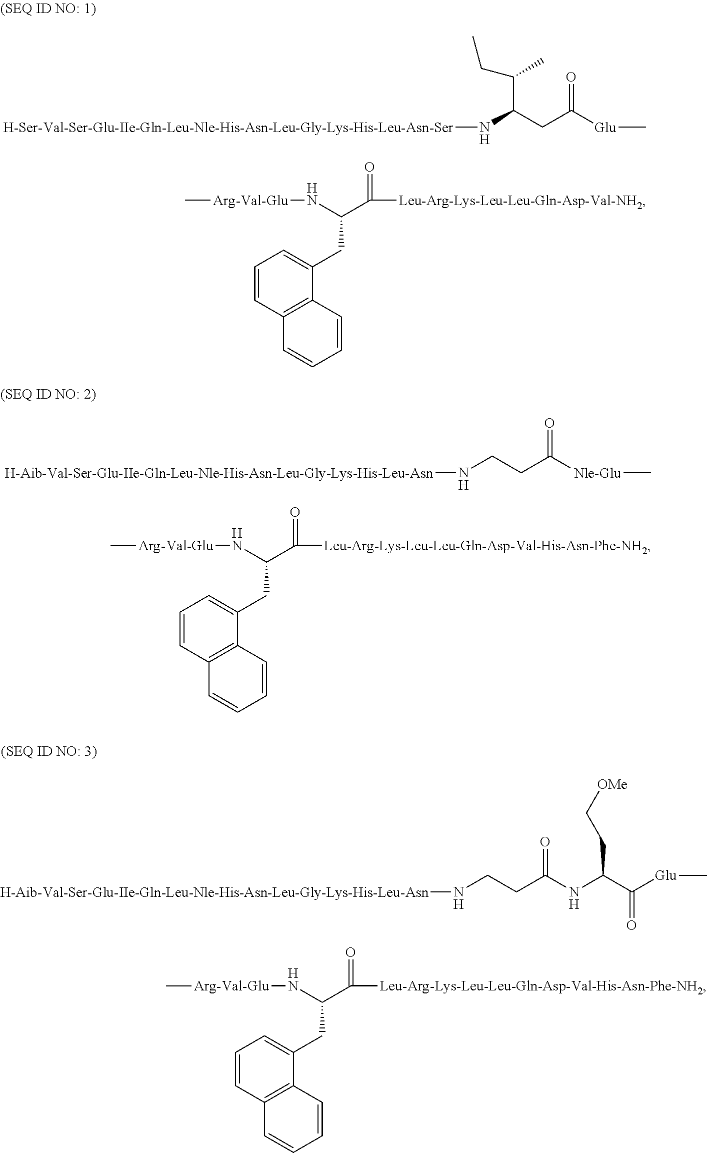

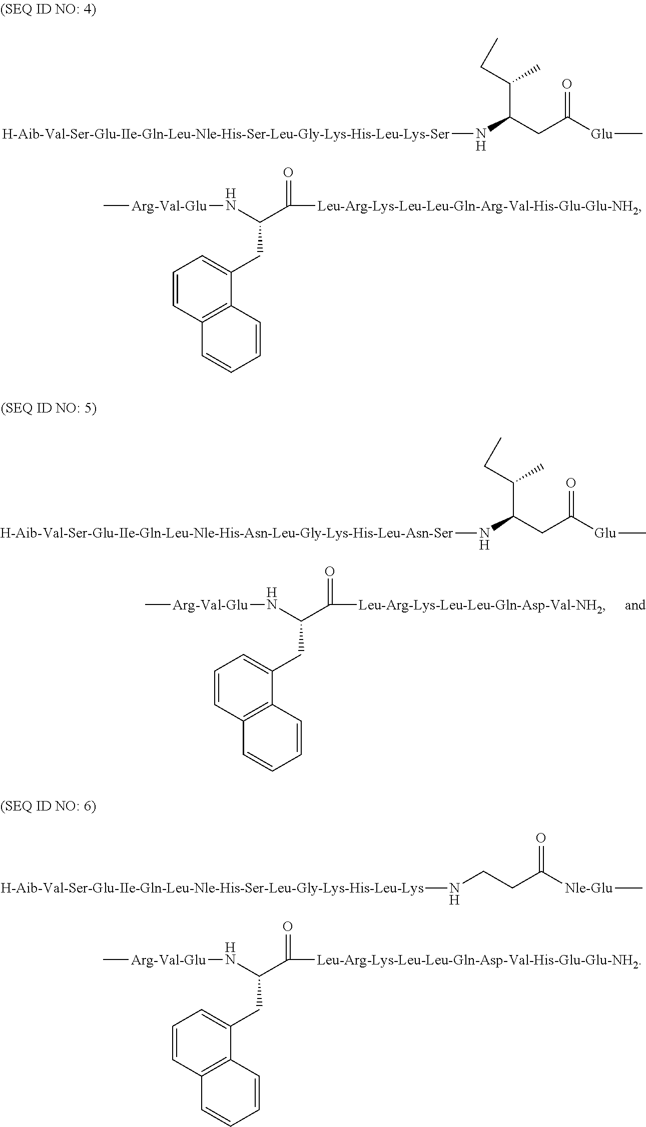

12. (canceled)

13. A pharmaceutical composition comprising a compound according to claim 1 as active ingredient in association with a pharmaceutically acceptable adjuvant, diluent or carrier.

14.-16. (canceled)

17. A method of one of the group consisting of:
treatment of psoriasis;
treatment of hypoparathyroidism;
treatment of a disease of the bone;
enhancement of bone growth;
acceleration of bone growth;
treatment of a bone fracture; and
stimulating fixation of a surgical implant to a bone,
the method comprising administering to an animal patient, wherein an animal includes a human, of a therapeutically effective amount of a compound according to claim 1.

18. The method of claim 17 wherein the disease of the bone is selected from the group consisting of osteoporosis, rickets, osteomalacia and Paget's disease.

19. The method of claim 17 wherein the bone fracture is a cancellous bone fracture.

20. A pharmaceutical composition comprising a compound according to claim 11 as active ingredient in association with a pharmaceutically acceptable adjuvant, diluent or carrier.

21. A method of one of the group consisting of:
treatment of psoriasis;
treatment of hypoparathyroidism;
treatment of a disease of the bone;
enhancement of bone growth;
acceleration of bone growth;
treatment of a bone fracture; and
stimulating fixation of a surgical implant to a bone,
the method comprising administering to an animal patient, wherein an animal includes a human, of a therapeutically effective amount of a compound according to claim 11.


SEQUENCE LISTING (57 sequences)

SEQ ID NO: 1 - protein, 31 aa, Artificial Sequence
  Description of Artificial Sequence Synthetic polypeptide
SVSEIQLXHNLGKHLNSXERVEXLRKLLQDV

SEQ ID NO: 2 - protein, 34 aa, Artificial Sequence
  Description of Artificial Sequence Synthetic polypeptide
XVSEIQLXHNLGKHLNXXERVEXLRKLLQDVHNF

SEQ ID NO: 3 - protein, 34 aa, Artificial Sequence; identical to SEQ ID NO: 2
  Description of Artificial Sequence Synthetic polypeptide

SEQ ID NO: 4 - protein, 34 aa, Artificial Sequence
  Description of Artificial Sequence Synthetic polypeptide
XVSEIQLXHSLGKHLKSXERVEXLRKLLQRVHEE

SEQ ID NO: 5 - protein, 31 aa, Artificial Sequence
  Description of Artificial Sequence Synthetic polypeptide
XVSEIQLXHNLGKHLNSXERVEXLRKLLQDV

SEQ ID NO: 6 - protein, 34 aa, Artificial Sequence
  Description of Artificial Sequence Synthetic polypeptide
XVSEIQLXHSLGKHLKXXERVEXLRKLLQRVHEE

SEQ ID NO: 7 - protein, 34 aa, Artificial Sequence
  Description of Artificial Sequence Synthetic polypeptide
SVSEIQLXHNLGKHLNSXXRVEXLRKKLQDVHNF

SEQ ID NO: 8 - protein, 34 aa, Artificial Sequence
  Description of Artificial Sequence Synthetic polypeptide
SVSEIQLXHNLGKHLNSXERVEXLRKKLQDVHNF

SEQ ID NO: 9 - protein, 34 aa, Artificial Sequence
  Description of Artificial Sequence Synthetic polypeptide
SVSEIQLXHNLGKHLNSXERVEXLRKLLQDVHNF

SEQ ID NO: 10 - protein, 31 aa, Artificial Sequence; identical to SEQ ID NO: 1
  Description of Artificial Sequence Synthetic polypeptide

SEQ ID NO: 11 - protein, 34 aa, Artificial Sequence
  Description of Artificial Sequence Synthetic polypeptide
SVSEIQLXHNLGKHLNSXXRVEXLRKLLQDVHNF

SEQ ID NO: 12 - protein, 34 aa, Artificial Sequence
  Description of Artificial Sequence Synthetic polypeptide
AVSEIQLXHNLGKHLNXXERVEXLRKKLQDVHNF

SEQ ID NO: 13 - protein, 34 aa, Artificial Sequence
  Description of Artificial Sequence Synthetic polypeptide
XVSEIQLXHNLGKHLNXXERVEXLRKKLQDVHNF

SEQ ID NO: 14 - protein, 34 aa, Artificial Sequence
  Description of Artificial Sequence Synthetic polypeptide
AVSEIQLXHNLGKHLNXXERVEXLRKLLQDVHNF

SEQ ID NO: 15 - protein, 34 aa, Artificial Sequence
  Description of Artificial Sequence Synthetic polypeptide
SVSEIQLXHNLGKHLNXXERVEXLRKLLQDVHNF

SEQ ID NO: 16 - protein, 34 aa, Artificial Sequence; identical to SEQ ID NO: 15
  Description of Artificial Sequence Synthetic polypeptide

SEQ ID NO: 17 - protein, 34 aa, Artificial Sequence; identical to SEQ ID NO: 15
  Description of Artificial Sequence Synthetic polypeptide

SEQ ID NO: 18 - protein, 34 aa, Artificial Sequence; identical to SEQ ID NO: 9
  Description of Artificial Sequence Synthetic polypeptide

SEQ ID NO: 19 - protein, 34 aa, Artificial Sequence; identical to SEQ ID NO: 9
  Description of Artificial Sequence Synthetic polypeptide

SEQ ID NO: 20 - protein, 34 aa, Artificial Sequence; identical to SEQ ID NO: 11
  Description of Artificial Sequence Synthetic polypeptide

SEQ ID NO: 21 - protein, 34 aa, Artificial Sequence
  Description of Artificial Sequence Synthetic polypeptide
SVSEIQLXHNLGKHLESXERVEXLRKLLQDVHNF

SEQ ID NO: 22 - protein, 34 aa, Artificial Sequence
  Description of Artificial Sequence Synthetic polypeptide
SVSEIQLXHNLGKHLSSXERVEXLRKLLQDVHNF

SEQ ID NO: 23 - protein, 34 aa, Artificial Sequence
  Description of Artificial Sequence Synthetic polypeptide
SVSEIQLXHSLGKHLNSXERVEXLRKLLQDVHNF

SEQ ID NO: 24 - protein, 34 aa, Artificial Sequence
  Description of Artificial Sequence Synthetic polypeptide
SVSEIQLXHELGKHLNSXERVEXLRKLLQDVHNF

SEQ ID NO: 25 - protein, 34 aa, Artificial Sequence
  Description of Artificial Sequence Synthetic polypeptide
SVSEIQLXHNLGKHLNSXERVEXLRKLLQHVHNF

SEQ ID NO: 26 - protein, 34 aa, Artificial Sequence
  Description of Artificial Sequence Synthetic polypeptide
SVSEIQLXHNLGKHLNSXERVEXLRKLLQSVHNF

SEQ ID NO: 27 - protein, 34 aa, Artificial Sequence
  Description of Artificial Sequence Synthetic polypeptide
SVSEIQLXHNLGKHLNSXERVEXLRKLLQDVHEF

SEQ ID NO: 28 - protein, 34 aa, Artificial Sequence
  Description of Artificial Sequence Synthetic polypeptide
SVSEIQLXHNLGKHLNSXERVEXLRKLLQDVHKF

SEQ ID NO: 29 - protein, 34 aa, Artificial Sequence
  Description of Artificial Sequence Synthetic polypeptide
SVSEIQLXHNLGKHLNSXERVEXLRKLLQDVHNE

SEQ ID NO: 30 - protein, 34 aa, Artificial Sequence
  Description of Artificial Sequence Synthetic polypeptide
SVSEIQLXHNLGKHLNSXERVEXLRKLLQDVHNK

SEQ ID NO: 31 - protein, 34 aa, Artificial Sequence
  Description of Artificial Sequence Synthetic polypeptide
SVSEIQLXHNLXXHLNSXERVEXLRKLLQDVHNF

SEQ ID NO: 32 - protein, 34 aa, Artificial Sequence
  Description of Artificial Sequence Synthetic polypeptide
SVSEIQLXHNLXKHLNSXERVEXLRKLLQDVHNF

SEQ ID NO: 33 - protein, 34 aa, Artificial Sequence
  Description of Artificial Sequence Synthetic polypeptide
SVSEIQLXHNLGXHLNSXERVEXLRKLLQDVHNF

SEQ ID NO: 34 - protein, 34 aa, Artificial Sequence
  Description of Artificial Sequence Synthetic polypeptide
SVSEIQLXHNLGKHLKSXERVEXLRKLLQDVHNF

SEQ ID NO: 35 - protein, 34 aa, Artificial Sequence
  Description of Artificial Sequence Synthetic polypeptide
SVSEIQLXHNLGKHLNSXERVEXLRKLLQRVHNF

SEQ ID NO: 36 - protein, 34 aa, Artificial Sequence
  Description of Artificial Sequence Synthetic polypeptide
XVSEIQLXHNLGKHLNSXERVEXLRKLLQDVHNF

SEQ ID NO: 37 - protein, 34 aa, Artificial Sequence
  Description of Artificial Sequence Synthetic polypeptide
SVSEIQLXHNLGKHLKSXERVEXLRKLLQRVHNF

SEQ ID NO: 38 - protein, 34 aa, Artificial Sequence
  Description of Artificial Sequence Synthetic polypeptide
XVSEIQLXHSLGKHLKSXERVEXLRKLLQRVHEF

SEQ ID NO: 39 - protein, 34 aa, Artificial Sequence
  Description of Artificial Sequence Synthetic polypeptide
XVSEIQLXHNLGKHLKSXERVEXLRKLLQRVHEF

SEQ ID NO: 40 - protein, 34 aa, Artificial Sequence
  Description of Artificial Sequence Synthetic polypeptide
SVSEIQLXHSLGKHLKSXERVEXLRKLLQRVHEF

SEQ ID NO: 41 - protein, 34 aa, Artificial Sequence
  Description of Artificial Sequence Synthetic polypeptide
SVSEIQLXHNLGKHLKSXERVEXLRKLLQRVHEF

SEQ ID NO: 42 - protein, 34 aa, Artificial Sequence; identical to SEQ ID NO: 36
  Description of Artificial Sequence Synthetic polypeptide

SEQ ID NO: 43 - protein, 34 aa, Artificial Sequence; identical to SEQ ID NO: 36
  Description of Artificial Sequence Synthetic polypeptide

SEQ ID NO: 44 - protein, 34 aa, Artificial Sequence
  Description of Artificial Sequence Synthetic polypeptide
SVSEIQLXHSLGKHLKSXERVEXLRKLLQRVHEE

SEQ ID NO: 45 - protein, 31 aa, Artificial Sequence
  Description of Artificial Sequence Synthetic polypeptide
XVSEIQLXHSLGKHLKSXERVEXLRKLLQRV

SEQ ID NO: 46 - protein, 34 aa, Artificial Sequence; identical to SEQ ID NO: 9
  Description of Artificial Sequence Synthetic polypeptide

SEQ ID NO: 47 - protein, 34 aa, Artificial Sequence
  Description of Artificial Sequence Synthetic polypeptide
XVSEIQLXHNLGKHLNSXXRVEXLRKLLQDVHNF

SEQ ID NO: 48 - protein, 34 aa, Artificial Sequence; identical to SEQ ID NO: 2
  Description of Artificial Sequence Synthetic polypeptide

SEQ ID NO: 49 - protein, 31 aa, Artificial Sequence
  Description of Artificial Sequence Synthetic polypeptide
SVSEIQLXHSLGKHLKSXERVEXLRKLLQRV

SEQ ID NO: 50 - protein, 34 aa, Artificial Sequence
  Description of Artificial Sequence Synthetic polypeptide
XVSEIQLXHELGKHLKSXERVEXLRKLLQEVHEK

SEQ ID NO: 51 - protein, 34 aa, Homo sapiens
SVSEIQLMHNLGKHLNSMERVEWLRKKLQDVHNF

SEQ ID NO: 52 - protein, 34 aa, Artificial Sequence; identical to SEQ ID NO: 8
  Description of Artificial Sequence Synthetic polypeptide

SEQ ID NO: 53 - protein, 34 aa, Artificial Sequence; identical to SEQ ID NO: 8
  Description of Artificial Sequence Synthetic polypeptide

SEQ ID NO: 54 - protein, 34 aa, Artificial Sequence; identical to SEQ ID NO: 8
  Description of Artificial Sequence Synthetic polypeptide

SEQ ID NO: 55 - protein, 34 aa, Artificial Sequence; identical to SEQ ID NO: 13
  Description of Artificial Sequence Synthetic polypeptide

SEQ ID NO: 56 - protein, 34 aa, Artificial Sequence
  Description of Artificial Sequence Synthetic polypeptide
XVSEIQLXHXLXXHLXXXXRVEXLRKXLQXVXXX

SEQ ID NO: 57 - protein, 34 aa, Artificial Sequence; identical to SEQ ID NO: 56
  Description of Artificial Sequence Synthetic polypeptide